(19) United States
(12) Patent Application Publication
(10) Pub. No.: US 2020/0129544 A1
(43) Pub. Date: Apr. 30, 2020

(54) IRON GLYCINE SULFATE COMPOSITIONS AND USES THEREOF

(71) Applicant: ROZEGEN LLC, Albany, NY (US)

(72) Inventor: Chao-Yang Gau, Albany, NY (US)

(21) Appl. No.: 16/623,925

(22) PCT Filed: Jun. 29, 2018
(86) PCT No.: PCT/US2018/040212

§ 371 Date: Dec. 18, 2019

Related U.S. Application Data
(60) Provisional application No. 62/527,170, filed on Jun. 30, 2017.

(51) Int. Cl. A61K 33/26; A61K 31/197

(52) U.S. Cl. CPC A61K 33/26; A61K 31/197

(57) ABSTRACT

Compositions containing iron, glycine, and sulfate are provided.

### CROSS-REFERENCE TO RELATED APPLICATION

This application claims the benefit of, and priority to, U.S. Provisional Patent Application No. 62/527,170, filed Jun. 30, 2017, the contents of which are incorporated by reference.

### FIELD OF THE INVENTION

This application related to iron glycine sulfate compositions and uses thereof.

### BACKGROUND

Many diseases are associated with aberrant metabolism of nitric oxide (NO) and/or sulfate, including cancer, neurodegenerative disorders, such as Parkinson's and Alzheimer's diseases, inflammatory disorders, such as arthritis, and metabolic disorders, such as diabetes. Each year millions of people are newly diagnosed with one of those diseases in the United States alone. Such diseases claim the lives of many patients and cause others to become progressively incapacitated. The suffering extends to patients' families and social circles, who have to deal with the loss of a loved one or find ways to care for individuals who may need temporary or even permanent care. In addition, such diseases exact a financial toll. For example, the estimated direct medical costs for cancer treatment in the United States in 2014 were $87.8 billion, and some sources project that this number could exceed $200 billion by 2020.

Nitric oxide is an unstable gas that acts as a signaling molecule in variety of physiological processes. Laboratory evidence suggests that increased levels of NO may be useful in combating diseases associated with altered NO metabolism, but mechanisms for raising NO levels in a patient are lacking. Because NO is a gas, it is difficult to administer to patients in a controlled manner that achieves the desired levels in targeted tissues. Nitric oxide is synthesized in the body by nitric oxide synthase (NOS) enzymes. However, depending on their chemical environment, NOSs can also produce other reactive molecules that have harmful effects, so altering NOS activity or expression is challenging and risky. Consequently, existing treatments fail to treat abnormal NO metabolism directly, and people continue to suffer and die from diseases like cancer, Parkinson's disease, and Alzheimer's disease each year.

### SUMMARY

The invention provides therapeutic compositions containing non-chelated iron, glycine, and sulfate. The compositions may include an iron-glycine-sulfate compound in a pentahydrate, crystallized form. The compositions may also include ferrous sulfate in various states of hydration, such as monohydrate or tetrahydrate, glycine in various crystalline forms, such as alpha-form or gamma-form, arginine, or citrulline. Because NOSs synthesize NO from arginine using heme-bound iron as a catalyst, the compositions may promote synthesis of NO in the body. Also provided are methods of treating conditions using the compositions.

The therapeutic compositions and methods are useful for the treatment of a variety of conditions that can be ameliorated by increased production of NO by NOSs. Examples of conditions that respond to elevated NO levels includes cancers, neurological disorders, immune diseases, inflammatory diseases, and metabolic disorders. Administration of the compositions of the invention can result in improvement of these conditions, thereby reducing the burden of such diseases in both human and financial terms.

In an aspect of the invention, the compositions may include a compound of formula (I):

[M(C₂H₅NO₂)₂(H₂O)₄][M(H₂O)₆](SO₄)₂, (I)

in which M is a metal, and the [M(C₂H₅NO₂)₂(H₂O)₄] and the [M(H₂O)₆] groups are linked to each other via a SO₄ that forms a bond with an H₂O molecule from each of the [M(C₂H₅NO₂)₂(H₂O)₄] and the [M(H₂O)₆] groups. The metal in the compound is not chelated by (C₂H₅NO₂) or SO₄. The metal may have an oxidation state of +2. The metal may be Ca, Co, Cr, Cu, Fe, Mg, Mn, Ni, Se, or Zn. Alternatively, the metal may be a metal other than Co, Mg, Mn, Ni, and Zn. The metal may be Fe.

In another aspect, the invention includes compositions that include (1) a compound of formula (II):

X.MSO₄.5H₂O (II)

in which X is an amino acid-containing component, such as an amino acid, peptide, or protein, and M is a metal that is not chelated by X or SO₄, and (2) one or more of X, MSO₄.4H₂O, or MSO₄.H₂O. The metal may have an oxidation state of +2. The metal may be Ca, Co, Cr, Cu, Fe, Mg, Mn, Ni, Se, or Zn. Preferably, the metal is Fe. The metal may be Fe, and MSO₄.4H₂O and MSO₄.H₂O may be ferrous sulfate tetrahydrate and ferrous sulfate monohydrate, respectively. Preferably, the amino acid is glycine. Glycine may be provided as crystals in alpha-form, gamma-form, or both. The composition may include arginine or citrulline.

In another aspect, the invention includes compositions that include (1) a compound comprising a metal, an amino acid-containing component, and sulfate, and (2) arginine or citrulline. Preferably, the metal in the compound is not chelated by the amino acid or the sulfate. The metal may have an oxidation state of +2. The metal may be Ca, Co, Cr, Cu, Fe, Mg, Mn, Ni, Se, or Zn. Preferably, the amino acid-containing component is glycine. The composition may include one or more other compounds. The other compound may be a sulfate of the metal in a hydrated form. For example, the metal may be iron, and the metal sulfate may be ferrous sulfate tetrahydrate or ferrous sulfate monohydrate. The other compound may be the amino acid-containing component in a crystalline form. For example, the amino acid may be glycine, and the glycine may be in alpha-form or gamma-form.

In another aspect, the invention provides methods of treating a condition. The methods include providing a composition that includes a compound that includes a metal, an amino acid-containing component, such as an amino acid, peptide, or protein, and sulfate, in which the metal is not chelated by the amino acid-containing component or the sulfate. The compound may have a formula (II):

X.MSO₄.5H₂O (II)

in which M is a metal and X is the amino acid-containing component. The metal may have an oxidation state of +2. The metal may be Ca, Co, Cr, Cu, Fe, Mg, Mn, Ni, Se, or Zn. The amino acid-containing component may be glycine.

The composition may include one or more other compounds. The other compound may be a sulfate of the metal in a hydrated form. For example, the metal may be iron, and the metal sulfate may be ferrous sulfate tetrahydrate or ferrous sulfate monohydrate. The other compound may be the amino acid-containing component in a crystalline form. For example, the amino acid-containing component may be glycine, and the glycine may be in alpha-form or gamma-form. The other compound may be arginine or citrulline.

The condition may be cancer, a neurological disorder, an immune disease, an inflammatory disease, or a metabolic disorder. The cancer may be cancer of the breast, colon, lung, liver, pancreas, cervix, head and neck, gastric system, or brain. The neurological disorder may be Alzheimer's Disease, Parkinson's disease, or an autism spectrum disorder. The inflammatory disorder may be arthritis, such as rheumatoid arthritis or osteoarthritis. The metabolic disorder may be diabetes. The condition may be osteoporosis.

The composition may stimulate or attenuate a physiological process. For example, the composition may stimulate or attenuate nitric oxide production, NOS activity, cell proliferation, cell migration, angiogenesis, sensitivity to chemotherapeutic agents, sensitivity to radiation, sensitivity to insulin, liver function, sulfate retention, or immune response to a bacterial infection, viral infection, vaccine, or other immunogenic agent. The composition may alter levels of a chemical or molecule in a body fluid. The chemical or molecule may be nitric oxide, superoxide (O₂⁻) and peroxynitrite (ONOO^{-),} sulfate, glucose, cholesterol, a triglyceride, low-density lipoprotein, aspartate aminotransferase (AST), or alanine aminotransferase (ALT). The body fluid may be saliva, urine, blood, plasma, serum, semen, feces, or phlegm.

In another aspect, the invention provides methods of increasing NO production in a subject. The methods include providing a subject with any composition of the invention, as described above. NO levels may be measured in a body fluid, such as saliva, urine, blood, plasma, serum, semen, feces, or phlegm.

In another aspect, the invention provides methods increasing sulfate retention in a subject. The methods include providing a subject with any composition of the invention, as described above. Retention of sulfate can determined from levels of sulfate excreted in a body fluid, such as urine or feces.

In another aspect, the invention provides methods of inhibiting growth of cancer cells. The methods include administering to cells any composition of the invention, as described above. The composition may be administered at a concentration of about 0.1 mg/ml, 0.25 mg/ml, 0.5 mg/ml, or 1 mg/ml. The cells may be from any type of cancer, such as pancreatic cancer or lung cancer. The cancer cells may be from a tumor of epithelial tissue, such as an adenocarcinoma.

In another aspect, the invention provides compositions for use in treating a condition. The composition may be any composition of the invention, as described above. The condition may be cancer, a neurological disorder, an immune disease, an inflammatory disease, or a metabolic disorder. The cancer may be cancer of the breast, colon, lung, liver, pancreas, cervix, head and neck, gastric system, or brain. The neurological disorder may be Alzheimer's disease, Parkinson's disease, or an autism spectrum disorder. The inflammatory disorder may be arthritis, such as rheumatoid arthritis or osteoarthritis. The metabolic disorder may be diabetes. The condition may be osteoporosis.

In another aspect the invention includes methods of improving athletic performance in a subject. The methods include providing a subject with any composition of the invention, as described above. Athletic performance may include one or more of a subject's strength, endurance, speed, quickness, balance, jumping ability, ability to throw or shoot a ball for distance or accuracy, or ability to hit a ball with an athletic device, such as a bat, racket, golf club, for distance or accuracy.

### BRIEF DESCRIPTION OF THE DRAWINGS

FIG. 1 illustrates an amino acid-metal-sulfate-water composition according to an embodiment of the invention.

FIG. 2 illustrates an amino acid-metal-sulfate-water composition according to an embodiment of the invention.

FIG. 3 illustrates the crystal structure of tetra aqua diglycine ferrous hexa aqua ferrous bis sulfate according to a composition of the invention.

FIG. 4 illustrates a potential mechanism by which compounds of the invention may be selectively targeted to cancer cells.

FIG. 5 illustrates the synthesis of nitric oxide by NOS, which may be facilitated by the compounds of the invention.

FIG. 6 illustrates a mechanism by which a composition of the invention may alter sulfate metabolism to confer therapeutic benefits.

FIG. 7 illustrates a method of treating a subject with a composition according to an embodiment of the invention.

FIG. 8 is a graph showing nitric oxide levels in saliva at after administration of compositions of the invention.

FIG. 9 is a graph showing sulfate concentration in urine after administration of compositions of the invention.

FIG. 10 is a graph showing the growth rate of BEAS-2B cells cultured in the presence of a composition of the invention at various concentrations.

FIG. 11 is a graph showing the growth rate of PC-9 cells cultured in the presence of a composition of the invention at various concentrations.

FIG. 12 is a graph showing the growth rate of AsPC-1 cells cultured in the presence of a composition of the invention at various concentrations.

FIG. 13 is a graph showing the growth rate of BxPC-3 cells cultured in the presence of a composition of the invention at various concentrations.

FIG. 14 is graph showing results from electrospray mass spectrometric (ES-MS) analysis of a monomeric form of TAD-600.

FIG. 15 is graph showing results from ES-MS analysis of a dimeric form of TAD-600.

FIG. 16 is graph showing results from ES-MS analysis of a trimeric form of TAD-600.

FIG. 17 shows the effect of different samples of TAD-600 on inhibition of growth of AsPC-1 cells.

FIG. 18 shows the effect of different samples of TAD-600 on inhibition of growth of BxPC-3 cells.

FIG. 19 shows the effect of different samples of TAD-600 on inhibition of growth of PC-9 cells.

### DETAILED DESCRIPTION

The invention provides compositions and methods for treating chronic conditions that respond to increased levels of nitric oxide (NO). NO is a short-lived, endogenously-produced gas that acts as a signaling molecule in variety of physiological processes. NO manifests its biological actions via a wide range of chemical reactions, which depend on NO concentration and variations in the composition of intra- and extracellular milieu. Evidence indicates that NO may play a salutary role in treating diseases such as cancer and neurodegenerative diseases. See, e.g., Choudhari, S. K. et al., Nitric oxide and cancer: a review, World J Surg. Oncol. 11:118 (2013); Vahora, H. The Potential Role of Nitric Oxide in Halting Cancer Progression Through Chemoprevention, J. Cancer Prev, 21:1-12 (2016); Austin, S. A. et al., Endothelial Nitric Oxide Modulates Expression and Processing of Amyloid Precursor Protein, Circ. Res. 107:1498 (2010). However, because NO is a gas, it is difficult to administer to patients in a controlled manner that achieves the desired levels in targeted tissues. Provided herein are compositions that are stable in solid form and can be administered to patients to allow the body's natural enzymatic machinery to produce NO.

NO is synthesized by nitric oxide synthases (NOSs). Humans have three NOS genes: NOS1, NOS2, and NOS3. NOS1 and NOS3 are constitutively expressed in neuronal and endothelial cells, respectively, and are thus also known as nNOS and eNOS. In contrast, expression of NOS2 can be induced in most nucleated cell types, and this isoform is also called iNOS. NOSs catalyze the conversion of arginine to NO and citrulline via a mechanism that requires five cofactors, including heme and tetrahydrobiopterin (BH₄). However, NOSs can also produce superoxide (O₂) and peroxynitrite (ONOO⁻⁾ under appropriate environmental conditions, such as a low ratio of tetrahydrobiopterin:dihydropterin (BH₄:BH₂) or low concentration of arginine. Abnormal levels of NOS protein, NOS activity, or NO are associated with a variety of diseases and conditions, including cancers of the breast, colon, lung, liver, pancreas, cervix, head and neck, gastric system, and brain, Alzheimer's disease, Parkinson's disease, and diabetes.

The invention provides compositions that can promote NO production to treat conditions associated with low levels of NO or abnormal activity of NOSs. The invention includes compounds of the formula (I):

[M(C₂H₅NO₂)₂(H₂O)₄][M(H₂O)₆](SO₄)₂ (I),

in which M is a metal and the [M(C₂H₅NO₂)₂(H₂O)₄] and the [M(H₂O)₆] groups are linked to each other via a SO₄ that forms a bond with an H₂O molecule from each of the [M(C₂H₅NO₂)₂(H₂O)₄] and the [M(H₂O)₆] groups. Preferably, the metal in the compound is not chelated by (C₂H₅NO₂) or SO₄. The metal may have an oxidation state of +2. The metal may be Ca, Co, Cr, Cu, Fe, Mg, Mn, Ni, Se, or Zn. Alternatively, the metal may be a metal other than Co, Mg, Mn, Ni, and Zn. Preferably, the metal is Fe. Thus, in a preferred embodiment, the compound may be tetra aqua diglycine ferrous hexa aqua ferrous bis sulfate (condensed structural formula [Fe(C₂H₅NO₂)₂(H₂O)₄] [Fe(H₂O)₆](SO₄)₂; molecular formula C₄H₃₀Fe₂N₂O₂₂S₂; formula weight 634.12).

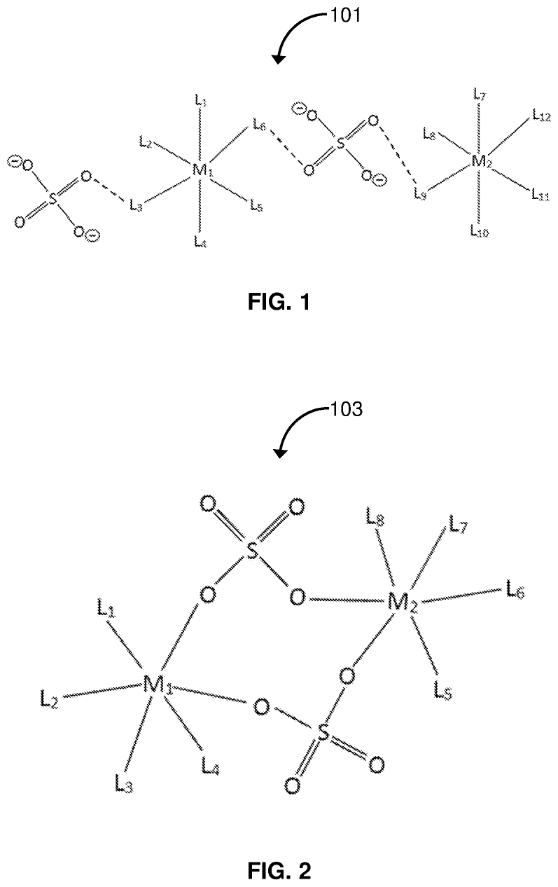

FIG. 1 illustrates an amino acid-metal-sulfate-water composition 101 according to an embodiment of the invention. The composition 101 is a supramolecule represented by formula (III):

[M_{I}(L_{I})ₙ(H₂O)₆₋ₙ][M₂(L_{II})ₘ(H₂O)₆₋ₘ](SO₄)₂ (III),

in which M_{I} and M_{II} are metal ions; L_{I} and L_{II} are ligands of water molecules or zwitterionic amino acids; n=0-6; and m=0-6. The zwitterionic amino acids have carboxyl, amino, carbonyl, or hydroxyl groups. For example and without limitation, the metal ions may be Fe, Cu, Zn, Mn, Ca, Mg, Ni, Cr, Se, or Co, and the zwitterionic amino acids may be alanine, arginine, asparagine, aspartic acid, citrulline, cysteine, glutamic acid, glutamine, glycine, histidine, isoleucine, leucine, lysine, methionine, phenylalanine, proline, serine, threonine, tryptophan, tyrosine, or valine. This class of compounds involves the coordination of amino acid and water molecules around the metal ion through a negatively charged oxygen atom on the carboxyl group. In addition, the positively charged nitrogen atoms on the amine group are not bonded and thus adjust the net charge of the coordination molecule. The negatively-charged sulfate tetrahedral molecules form strong hydrogen bonds with water and amino acid molecules that are coordinated by the metal ion. In addition, hydrogen bonds among amino acid molecules are formed. The three-dimensional network of hydrogen bounds connects heterogeneous molecules to form a supramolecular structure that exhibits biochemical and biophysical properties distinct from those of the individual molecules.

FIG. 2 illustrates an amino acid-metal-sulfate-water composition 103 according to an embodiment of the invention. The composition 103 is an amino acid-metal-sulfate-water dimer is represented by formula (IV):

[M_{I}(L_{I})ₙ(μ₂-SO₄)(H₂O)₄₋ₙ][M_{II}(L_{I})ₘ(μ₂-SO₄)(H₂O)₄₋ₘ] (IV),

in which M_{II} and M_{II} are metal ions; L_{I} and L_{II} are ligands of water molecules or zwitterionic amino acids; n=0-4; and m=0-4. The zwitterionic amino acids have carboxyl, amino, carbonyl, or hydroxyl groups. For example and without limitation, the metal ions may be Fe, Cu, Zn, Mn, Ca, Mg, Ni, Cr, Se, or Co, and the zwitterionic amino acids may be alanine, arginine, asparagine, aspartic acid, citrulline, cysteine, glutamic acid, glutamine, glycine, histidine, isoleucine, leucine, lysine, methionine, phenylalanine, proline, serine, threonine, tryptophan, tyrosine, or valine. This class of compounds contains dimers that include eight-membered rings formed by two sulfate tetrahedral structures and two octahedral metal structures, in which the ligands of the metal are two sulfate hydrogen atoms and four water or amino acid molecules. These rings are held together via hydrogen bounds between the water and amino acid molecules. The two metal octahedral complexes are bridged by two sulfate anions, forming a dimer structure.

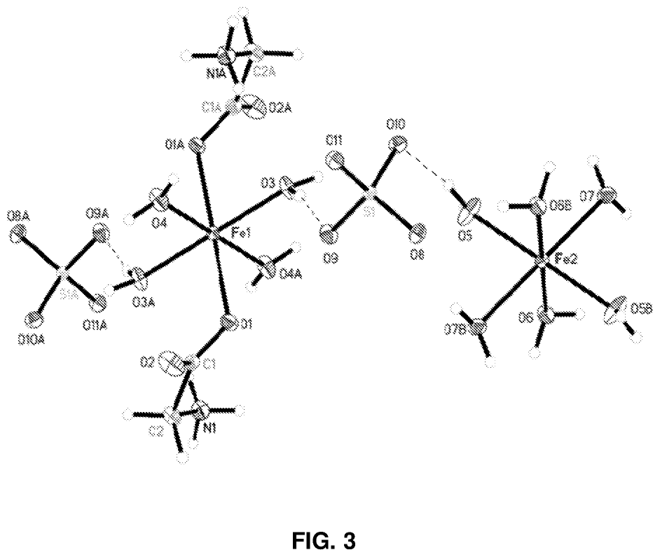

FIG. 3 illustrates the crystal structure of tetra aqua diglycine ferrous hexa aqua ferrous bis sulfate ([Fe(C₂H₅NO₂)₂(H₂O)₄][Fe(H₂O)₆](SO₄)₂) according to a composition of the invention. Tetra aqua diglycine ferrous hexa aqua ferrous bis sulfate is made by mixing solutions of ferrous sulfate and glycine, heating the mixture for a few hours, and allowing crystals of the compound to form.

In another aspect, the invention includes compositions that include (1) a compound of formula (II):

X.MSO₄.5H₂O (II),

in which X is an amino acid-containing component, such as an amino acid, peptide, or protein, and M is a metal that is not chelated by X or SO₄, and (2) one or more of X, MSO₄.4H₂O, or MSO₄.H₂O.

The metal may have an oxidation state of +2, such as cadmium, calcium, chromium, cobalt, copper, gold, iron, magnesium, manganese, molybdenum, nickel, platinum, scandium, silver, titanium, vanadium, and zinc. The metal may be Ca, Co, Cr, Cu, Fe, Mg, Mn, Ni, Se, or Zn. Preferably, the metal is Fe.

If the metal is iron, MSO₄.4H₂O is ferrous sulfate tetrahydrate (systematic name bis(μ-sulfato-O:O′)bis[tetraaquaferrous]; condensed structural formula [Fe(μ₂-SO₄)(H₂O)₄]₂; molecular formula Fe₂H₁₆O₁₆S₂; formula weight 447.95). If the metal is iron, MSO₄.H₂O is ferrous sulfate monohydrate, (condensed structural formula Fe(SO₄)(H₂O); formula weight 169.92).

Preferably, the amino acid-containing component is glycine. Glycine may be provided as crystals in alpha-form, gamma-form, or both.

In the first compound, the metal may be Ca, Co, Cr, Cu, Fe, Mg, Mn, Ni, Se, or Zn. Preferably, the metal is Fe. Preferably, the amino acid-containing component is glycine. Thus, the first compound may be represented by the formula (C₂H₅NO₂)FeSO₄.5H₂O.

The composition may include a compound of formula (II) and any number of the other compounds. Thus, if X is glycine and M is Fe, the composition may include one, two, three, or four of the following: ferrous sulfate tetrahydrate, ferrous sulfate monohydrate, alpha-form glycine, and gamma-form glycine.

The composition may include arginine or citrulline.

The invention includes compositions that include (1) a compound comprising a metal, an amino acid-containing component, and sulfate and (2) arginine or citrulline. Preferably, the metal in the compound is not chelated by the amino acid-containing component or the sulfate. Preferably, the metal has an oxidation state of +2. The metal may be Ca, Co, Cr, Cu, Fe, Mg, Mn, Ni, Se, or Zn. Preferably, the amino acid-containing component is glycine. The composition may include one or more of a sulfate of the metal in a hydrated form, such as a tetrahydrate or monohydrate, or the amino acid-containing component in a crystalline form. For example, the metal may be iron, and the metal sulfate may be ferrous sulfate tetrahydrate or ferrous sulfate monohydrate. The amino acid-containing component may be glycine, and the glycine may be in alpha-form or gamma-form.

Ferrous irons Fe(II) are unstable in water due to hydrolysis, and oxidize easily to the ferric Fe(III) state according to the following reactions:

Fe²⁺+2OH⁻->Fe(OH)₂ (A), and

2Fe(OH)₂+H₂O+½O₂->2Fe(OH)₃ (B).

Iron(II) hydroxide is moderately soluble in water, and iron(III) hydroxide is insoluble. In soluble iron(II) hydroxide and precipitates of both iron(II) hydroxide and iron(III) hydroxide, iron lies in the center of coordination sphere surrounded by water or hydroxide as ligands, as illustrated in formulas (V), (VI), and (VII):

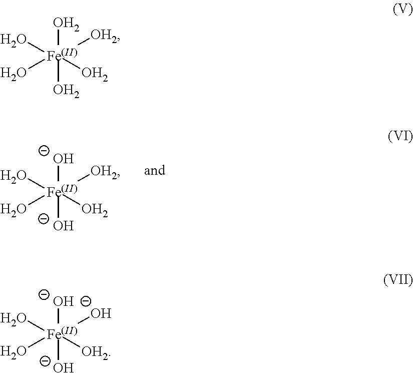

The neutral water ligands and negatively-charged hydroxide ligands form an interaction layer with other substances. In contrast, ferrous glycine aqueous molecules of the invention carry positive charges on the amine groups and presents a distinct electrostatic property when interacting with other substances, as illustrated in formula (VIII):

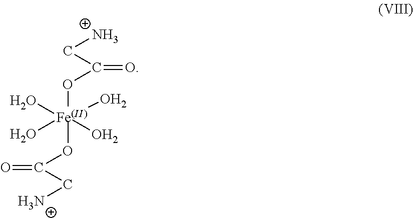

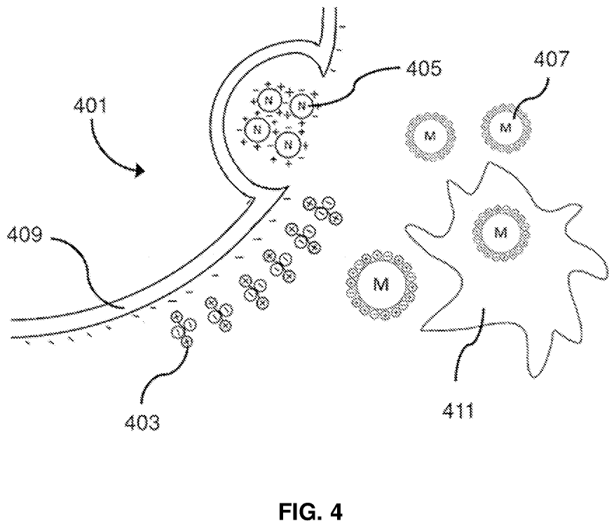

FIG. 4 illustrates a potential mechanism by which compounds of the invention may be selectively targeted to cancer cells. Without wishing to be bound by any particular theory, the positive charges on the exterior of metal-amino acid complexes may facilitate selective interactions between compositions of the invention and cancer cells. A cancer cell 401 produces lactate anions and sialic acid that causes its surface to have a higher net negative charge than the surface of normal primary cells. Consequently, cancer cells attract positively-charged particles more strongly. Ferrous glycine aqueous molecules 403 of the invention, which may exist as nanoparticles 405 or microparticles 407, may be selectively adsorbed onto the surface 409 of a cancer cell 401. Adsorption of ferrous glycine aqueous molecules 403 may in turn decrease the net negative charge on the surface of cancer cells, allowing such cells to be identified and destroyed by cells of the immune system, such as macrophages 411, without requiring a specific molecular biomarker.

Tetra aqua diglycine ferrous hexa aqua ferrous bis sulfate ([Fe(C₂H₅NO₂)₂(H₂O)₄][Fe(H₂O)₆](SO₄)₂) holds monomeric units together by strong hydrogen bonds and metal coordination, forming a three dimensional network of a supramolecular polymer with the formula [C₄H₃₀Fe₂N₂O₂₂S₂]ₙ with n≥2. Because the polymerization is based on noncovalent, dynamic and reversible interactions, [Fe(C₂H₅NO₂)₂(H₂O)₄] [Fe(H₂O)₆](SO₄)₂ has an advantageous combination of viscosity, bio-degradation, and bio-compatibility properties. On the other hand, ferrous sulfate monohydrate of has a network structure of Fe(II) and sulfate ions and forms suspensions when dissolved in water. Both [Fe(C₂H₅NO₂)₂(H₂O)₄][Fe(H₂O)₆](SO₄)₂ and ferrous sulfate monohydrate can be used to form particles with desired surface charge type and density based on positioning of amine, sulfate, hydroxyl, and carboxyl groups. By adjusting the ratio of the two compounds and/or the pH, particles of appropriate size and surface charge for different uses can be made.

Cellular uptake of small particles through endocytosis is governed by size and charge. Positively-charged particles are more attracted to negatively-charged membranes of cells and tissues, such as cancer cells, sarcomas, macrophages, bacteria, probiotics, endothelium, blood-brain barrier, mucus, bone marrow, fat cells, platelets, and cells with amyloid beta. Microparticles 407, typically having diameters from 0.1 μm to 100 μm, are capable of stimulating macrophages 411, leading to an inflammatory response of increased nitric oxide production and cytokine TNF-α release. Nanoparticles 405, typically having diameters from 1 nm to 100 nm, coated with positively charged amine group can facilitate selective uptake by endocytosis in cancer cells 401 and amplify the anticancer efficacy. In addition, positively-charged particles can neutralize the negative surface charge of the endothelial cell layer in the blood-brain barrier (BBB) and enhance barrier permeability, allowing effective drug delivery into the brain.

For example, TAD-600 is a zwitterion that includes cationic (e.g., amine) and anionic (e.g., carboxyl and sulfate) functional groups. The net charge of a TAD-600 molecule can be adjusted by the properties of the surrounding solution, e.g., pH value, O₂ concentration, and enzymatic activity. In acidic solutions, the molecules carry a positive net charge and aggregate to form large particles with high molecular weights, whereas in neutral or basic solutions, the molecules carry no net charge or a low net charge and disperse to form small particles with low molecular weights.

Many mixed-charged self-assembled zwitterionic small particles have been reported to interact strongly with the cell membrane, induce high endocytic uptake, increase accumulation in tumor and prevent protein/amyloid aggregation. Metallic zwitterionic particles may carry various metal ions to target at different diseases. For example, ferrous ion uptake in cells may change oxidative stress and lead to different ROS levels, which have been reported to inhibit cancer cell growth through ferroptosis. On the other hand, it is also reported that moderate ROS stimulation by ferrous ions may enhance antioxidants and protect neurons in various neurodegenerative diseases.

Without wishing to be bound by any particular theory, the compositions may exert beneficial effects by promoting synthesis of NO from arginine within the body.

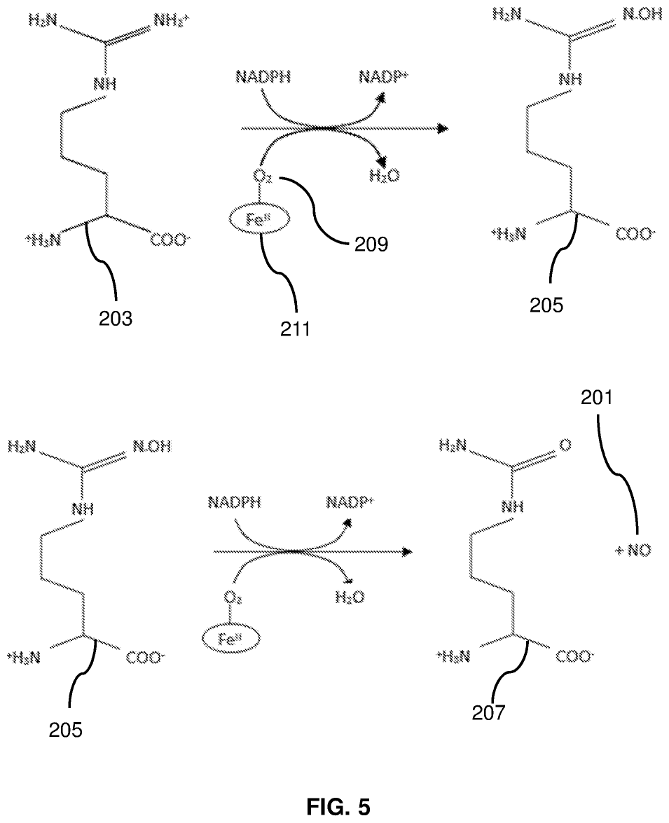

FIG. 5 illustrates the synthesis of NO 201 by NOS, which may be facilitated by the compounds of the invention. In the first step, arginine 203 (in the L form) is converted to N-hydroxy-L-arginine (NOHLA) 205. In the second step, NOHLA 205 is converted to citrulline 207 and NO 201. In each step, O₂ 209 serves as an electron acceptor. O₂ is provided by heme-bound Fe(II) 211, which is cofactor for NOS. The compositions may facilitate NO production by providing one or more of Fe(II), arginine, and citrulline.

Additionally or alternatively, the compositions may also provide therapeutic benefits by altering sulfate metabolism. Sulfate transport affects several essential chemicals, including cholesterol, glycosaminoglycan, protein, phenol, serotonin, dopamine, estrogen, testosterone, Vitamin D and melatonin. Sulfate is required for sulfation, the addition of sulfo groups to other molecules, by sulfotransferases. Sulfation is involved in a variety of biological processes, including detoxification, hormone regulation, molecular recognition, cell signaling, viral entry into cells, cell matrix synthesis, cell membrane maintenance, coagulation, hemostasis, fibrinolysis, angiogenesis, defense mechanism, endocytosis, apoptosis, cell recognition, cell proliferation, cell migration, and cell adhesion. Key biological molecules that are modified by sulfation include several glycosaminoglycans and polysaccharides of the extracellular matrix, such as heparin, heparan sulfate, chondroitin sulfate, and dermatan sulfate, as well as proteins and cholesterol. Sulfate and sulfate derivatives are known to control inflammatory responses at multiple levels, due to the relatively high negative charge density. For example, sulfo groups on heparan sulfate bind with many signaling molecules (e.g., chemokines, growth factors and cytokines) to lock them into an active site conformation and regulate the biological effect. Deficiency in sulfate causes physical deformities, mental retardation, pain and inflammation.

The major source of sulfate in the body is the oxidation of cysteine and methionine by sulfite oxidase. However, sulfate synthesis by oxidation of sulfur-containing amino acids requires superoxide (O₂), which causes inflammation. Sulfate may also be provided in the diet, but free anionic sulfate in food and water is believed to be poorly absorbed by gastrointestinal tract. The compositions of the invention, which contain sulfate bound to metal ions, such as Fe²⁺, and amino acids, such as glycine, enriches sulfate bioavailability in body and enhances bioactivity in sulfation.

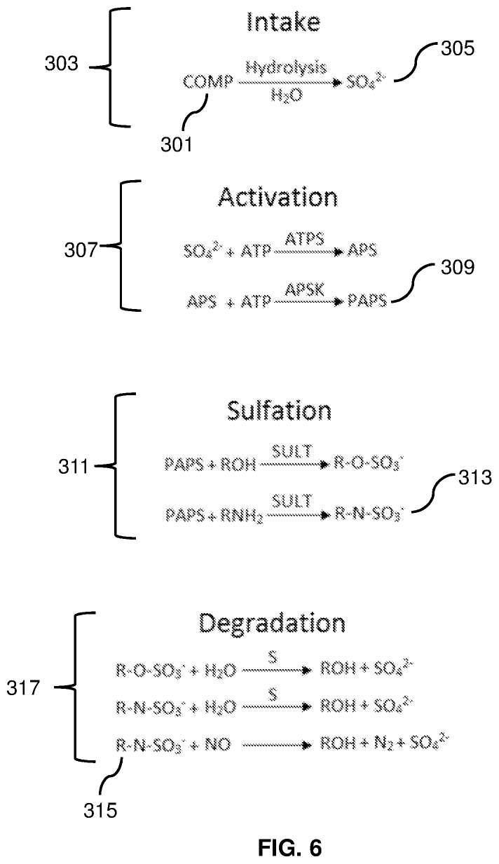

FIG. 6 illustrates a mechanism by which a composition 301 of the invention may alter sulfate metabolism to confer therapeutic benefits. In a hydrolysis or intake step 303, the composition 301 is hydrolyzed to produce free sulfate 305. In an activation step 307, 3′-phosphate 5′-sulfatophosphate (PAPS) 309 is generated in a series of ATP-dependent reactions. In a sulfation step 311 a sulfotransferase conjugates a sulfo group 313 to an oxygen or nitrogen atom of a biomolecule 315, such as a proteoglycan, polysaccharide, or protein. In a degradation step 317, the sulfo group 313 is removed from the biomolecule in a reaction with water or NO.

Additionally or alternatively, the compositions may also provide therapeutic benefits by altering glycine metabolism. By increasing serum glycine concentration by two- to four-fold, glycine supplementation can promote activation of glycine receptors (GlyRs). GlyRs are glycine-gated chloride channels that influence calcium current into cells, facilitating biological effects of nitric-oxide modulation, anti-inflammation, anti-angiogenesis, and cytoprotection.

The effect of a ligand stimulation of GlyRs depends on the cellular context. In endothelial cells, glycine causes a hyperpolarization of cell membrane by increased Cl⁻ influx, generating a transmembrane potential gradient that drives an increase of calcium influx. An elevation in the intracellular free calcium concentration enhances eNOS expression and nitric oxide release. The constitutive low-level eNOS-derived nitric oxide plays a key role in the regulation of vascular tone and cardiovascular system, and abnormalities in its productions causes diseases such as hypertension, atherosclerosis and angiogenesis-associated disorders. However, in neurons, macrophages, and smooth muscle cells, the hyperpolarization caused by glycine-derived Cl⁻ influx impairs opening of voltage-dependent Ca²⁺ channels and prevents increases in Ca²⁺ concentration. In neurons, activation of GlyR by glycine inhibits neurotransmitter release. In cells of the immune system, GlyR activation attenuates the function of TNF-alpha, NF-kB, iNOS and reactive oxygen species, decreases hypoxic cell injury, and inhibits the inflammatory response. In some cell types, glycine also functions in maintaining nitric oxide levels by regulating the levels of eNOS and iNOs.

The invention provides pharmaceutical compositions containing one or more of the compounds described above. A pharmaceutical composition containing the compounds may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the compounds in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,166,452 and 4,265,874, to form osmotic therapeutic tablets for control release. Preparation and administration of compounds is discussed in U.S. Pat. No. 6,214,841 and U.S. Pub. 2003/0232877, incorporated by reference herein in their entirety.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin or olive oil.

An alternative oral formulation, where control of gastrointestinal tract hydrolysis of the compound is sought, can be achieved using a controlled-release formulation, where a compound of the invention is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Each compound may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Examples of such materials are cocoa butter and polyethylene glycols.

For topical use, creams, ointments, jellies, fast melt tablets, solutions or suspensions are suitable as are nebulized forms for pulmonary delivery. Topical application includes the use of mouth washes and gargles.

The compositions may be provided as part of a kit. The kit may include on or more of the compounds in a vessel. The compounds may be provided as solids, for example, as powders, or as solutions in an aqueous or organic medium. The kit may include a medium in which the compounds can be dissolved, suspended, diluted, diluted, combined, mixed, or otherwise prepared for administration.

The invention also provides methods of treating a condition using the compositions provided herein.

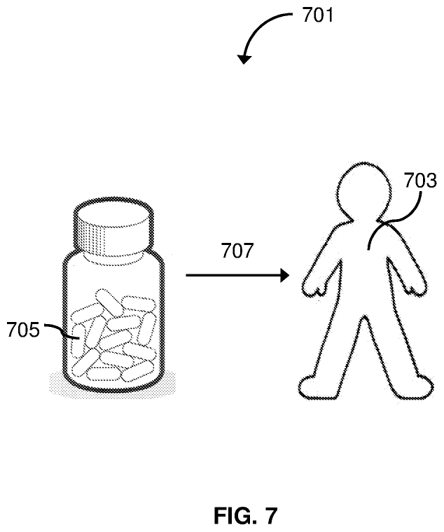

FIG. 7 illustrates a method 701 of treating a condition in a subject 703 with a composition 705 according to an embodiment of the invention. The composition 705 is provided 707 to the subject. The composition may be provided in any suitable form, for example, as a tablet, troche, lozenge, fast-melt, aqueous or oily suspension, dispersible powder or granule, emulsion, hard or soft capsule, syrup, or elixir.

Providing 707 may include administering the composition to the subject. The composition may be administered by any suitable means, such as oral, intravenous, enteral, parenteral, dermal, buccal, topical (including transdermal), injection, intravenous, nasal, pulmonary, and with or on an implantable medical device (e.g., stent or drug-eluting stent or balloon equivalents).

The condition may be a condition associate with abnormal levels of NOS protein, NOS activity, or NO. Abnormal NO metabolism has been detected in many cancer types, including cancers of the breast, colon, lung, liver, pancreas, cervix, head and neck, gastric system, and brain. Evidence on the role of NO in cancer symptoms and progression is conflicting. In some instances, increased NO has a cancer-promoting effect, whereas other evidence indicates that NO acts as an anti-cancer agent. See, e.g., Choudhari, S. K. et al., Nitric oxide and cancer: a review, World J Surg. Oncol. 11:118 (2013); and Vahora, H. The Potential Role of Nitric Oxide in Halting Cancer Progression Through Chemoprevention, J. Cancer Prev, 21:1-12 (2016). In addition, the effect of NO may depend on the level to which it is increased, with modest increases in NO concentration promoting proliferation of cancer cells and higher increases in NO concentration inhibiting proliferation. See, e.g., El-Sehemy, A. et al., Nitric oxide signaling in human ovarian cancer: A potential therapeutic target, Nitric Oxide 54:30-37 (2016); Wang, B. et al., A novel model system for studying the double-edged roles of nitric oxide production in pancreatic cancer growth and metastasis, Oncogene, 22:1771 (2003). However, the inventors have found that compounds of the invention specifically inhibit growth of cancerous cells at a range of concentrations but have little or no effect on the growth of normal cells at the same concentrations.

Other diseases and disorders are associated with abnormal NO levels, including neurological disorders, such as Alzheimer's disease, Parkinson's disease, or autism spectrum disorders, inflammatory disorders, such as arthritis (e.g., rheumatoid arthritis and osteoarthritis), metabolic disorders, such as diabetes, and osteoporosis. See, e.g., Austin, S. A. et al., Endothelial Nitric Oxide Modulates Expression and Processing of Amyloid Precursor Protein, Circ. Res. 107:1498 (2010); Austin, S. A. Supplementation of Nitric Oxide Attenuates ApPP and BACE1 Protein in Cerebral Microcirculation of eNOS-Deficient Mice, J. Alzheimer's Disease, 33:29-33 (2013).

The condition may be a condition associated with defects in sulfate metabolism. Sulfated proteoglycans, such as heparin sulfate, as associated with a variety of pathological conditions, including neurodegenerative diseases, such Parkinson's disease, Alzheimer's disease, Huntington's disease, amyotrophic lateral sclerosis, and multiple sclerosis. See, e.g., Zhang, X. Implications of heparan sulfate and heparanase in neuroinflammation, Matrix Biol. 25:174-181(2014).

The composition may be used to stimulate or attenuate a physiological process. For example, the composition may stimulate or attenuate nitric oxide production, NOS activity, cell proliferation, cell migration, angiogenesis, sensitivity to chemotherapeutic agents, sensitivity to radiation, sensitivity to insulin, liver function, sulfate retention, or immune response to a bacterial infection, viral infection, vaccine, or other immunogenic agent. The composition may alter levels of a chemical or molecule in a body fluid. The chemical or molecule may be nitric oxide, superoxide (O₂⁻) and peroxynitrite (ONOO⁻), sulfate, glucose, cholesterol, a triglyceride, low-density lipoprotein, aspartate aminotransferase (AST), or alanine aminotransferase (ALT). The body fluid may be saliva, urine, blood, plasma, serum, semen, feces, or phlegm.

The invention also provides methods of improving athletic performance by providing a subject with a composition of the invention. Athletic performance may include one or more of a subject's strength, endurance, speed, quickness, balance, jumping ability, ability to throw or shoot a ball for distance or accuracy, or ability to hit a ball with an athletic device, such as a bat, racket, golf club, for distance or accuracy. Any suitable measure of athletic performance may be used, such as timed distance runs, such as a 40-yard sprint, 100-yard sprint, 5K run, or 10K run, weight-lifting exercises, vertical jump tests, horizontal jump tests, the Functional Movement Screen, the Athletic Ability Assessment, and the like.

### Examples

Method of Making [Fe(C₂H₅NO₂)₂(H₂O)4][Fe(H₂O)₆](SO₄)₂.

Ferrous sulfate monohydrate or ferrous sulfate heptahydrate was mixed with glycine and water at an iron:glycine:water molar ratio from about (1):(1˜5):(0˜2). The mixture was heated from about 40° C. to 120° C. for 0.5 to 8 hours under humidity ranging from 0% to 60% with 0% to 25% air oxygen and then allowed to cool to room temperature. The solid fraction was dried to produce a mixture of ferrous sulfate tetrahydrate, ferrous sulfate monohydrate, alpha-form glycine, and gamma-form glycine. The solid mixture was dissolved in water at neutral or acidic pH by adding appropriate acid (e.g., hydrochloride, sulfuric acid, citric acid). The solution is then placed in the ambient environment with desiccant dehumidifiers and oxygen absorbers to produce [Fe(C₂H₅NO₂)₂(H₂O)₄][Fe(H₂O)₆](SO₄)₂, which may also exist in the previous solid fraction under various manufacturing conditions.

Example Crystal Paymasters.

The X-ray crystal diffraction pattern was analyzed for [Fe(C₂H₅NO₂)₂(H₂O)₄][Fe(H₂O)₆](SO₄)₂. The single-crystal X-ray diffraction obtained for [Fe(C₂H₅NO₂)₂(H₂O)₄][Fe(H₂O)₆](SO₄)₂ corresponds to a triclinic crystal of space group P-1, with the following cell dimensions: a=5.99 Å, b=6.78 Å, c=13.34 Å, α=85.45°, β=82.92°, and γ=83.09°. It will be understood that the values for crystal parameters were obtained from one sample of the compound and that the invention includes compounds with values that differ from those obtained. Effect of compositions on NO levels in saliva. The effects of compositions of the invention on NO levels in saliva were analyzed. Mixture 1 contains tetra aqua diglycine ferrous hexa aqua ferrous bis sulfate (condensed structural formula [Fe(C₂H₅NO₂)₂(H₂O)4] [Fe(H₂O)₆](SO₄)₂; molecular formula C₄H₃₀Fe₂N₂O₂₂S₂; formula weight 634.12), ferrous sulfate tetrahydrate (systematic name bis(μ-sulfato-O:O′)bis[tetraaquaferrous]; condensed structural formula [Fe(μ₂-SO₄)(H₂O)₄]₂; molecular formula Fe₂H₁₆O₁₆S₂; formula weight 447.95), ferrous sulfate monohydrate (condensed structural formula Fe(SO₄)(H₂O); formula weight 169.92), γ-glycine (condensed structural formula NH₂CH₂COOH; formula weight 75.07), and α-glycine condensed structural formula NH₂CH₂COOH; formula weight 75.07). Human subjects were given orally (1) a negative control, (2) Mixture 1, 65 mg total iron, (3) Mixture 1 plus 2 g arginine, 65 mg total iron, (4) 2 g arginine, or (5) FeSO₄ plus 2 g arginine, 65 mg total iron, and NO levels in saliva were measured at hourly intervals.

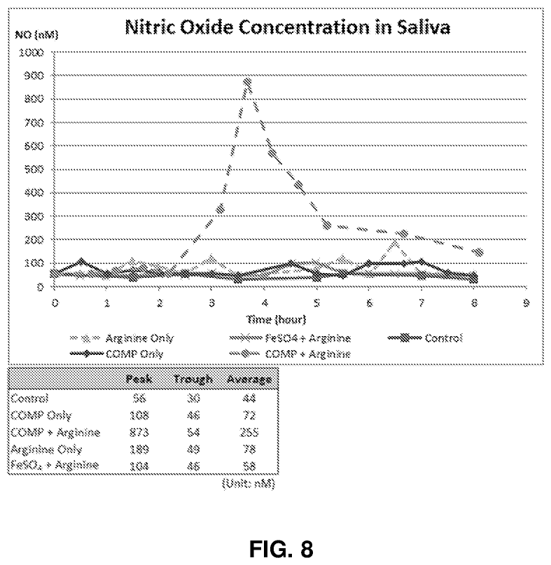

FIG. 8 is a graph showing NO levels in saliva at after administration of compositions of the invention. The table indicates the maximum (peak), minimum (trough), and mean (average) values for each condition. Administration of Mixture 1 plus arginine resulted in higher peak and average NO levels in saliva than did administration of either Mixture 1 alone, arginine alone, or FeSO₄ plus arginine.

Effect of Compositions on Sulfate Retention.

The effects of compositions of the invention on sulfate retention were analyzed. Human subjects were given orally (1) a negative control, (2) Mixture 1 plus 2 g arginine, 112 mg total sulfate, (3) 2 g arginine, or (4) FeSO₄ plus 2 g arginine, 112 mg total sulfate, and sulfate concentrations in urine were measured at hourly intervals.

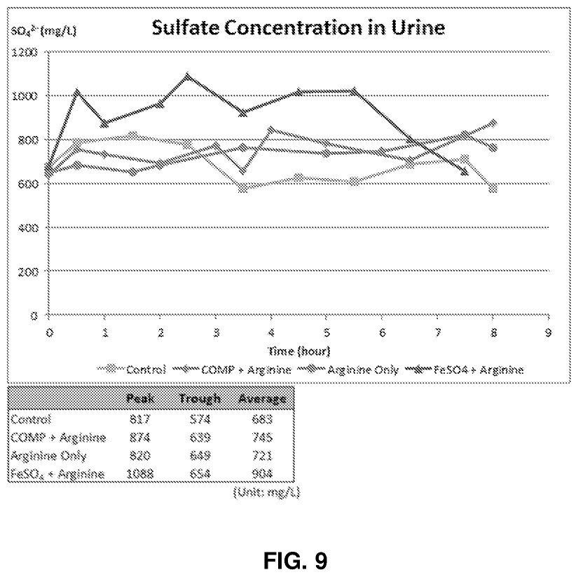

FIG. 9 is a graph showing sulfate concentration in urine after administration of compositions of the invention. The table indicates the maximum (peak), minimum (trough), and mean (average) values for each condition. Administration of Mixture 1 plus arginine resulted in lower concentration of sulfate in urine than did administration of ferrous sulfate plus arginine.

Effect of Compositions on Cell Growth.

The effects of compositions of the invention on cell growth were analyzed. Cells were cultured in the presence of various concentrations Mixture 1, and culture density was analyzed at daily intervals.

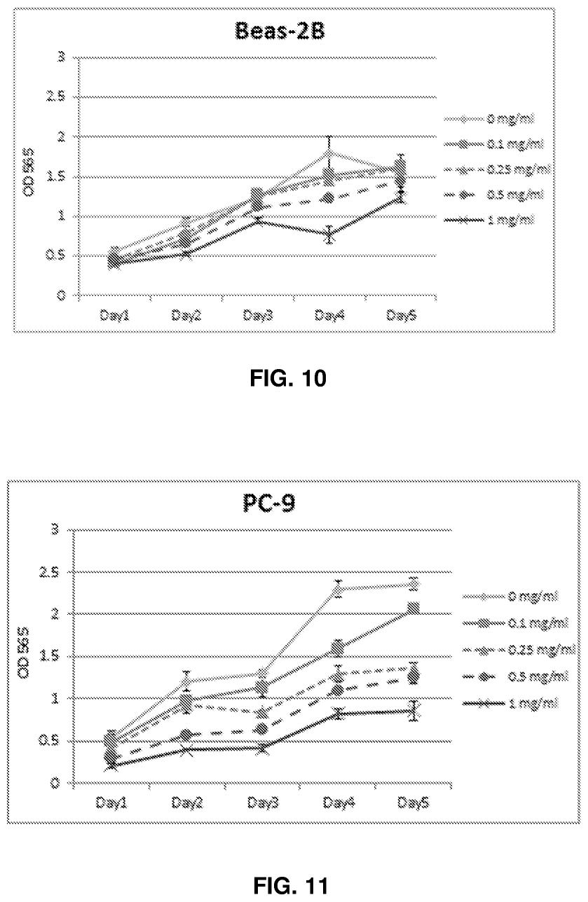

FIG. 10 is a graph showing the growth rate of BEAS-2B cells cultured in the presence of a composition of the invention at various concentrations. The BEAS-2B cell line is derived from normal, non-cancerous human bronchial epithelium. Mixture 1 had only a modest effect on growth of BEAS-2B cells.

FIG. 11 is a graph showing the growth rate of PC-9 cells cultured in the presence of a composition of the invention at various concentrations. The PC-9 cell line is derived from human lung adenocarcinoma. Mixture 1 inhibited growth of PC-9 cells in a concentration-dependent manner.

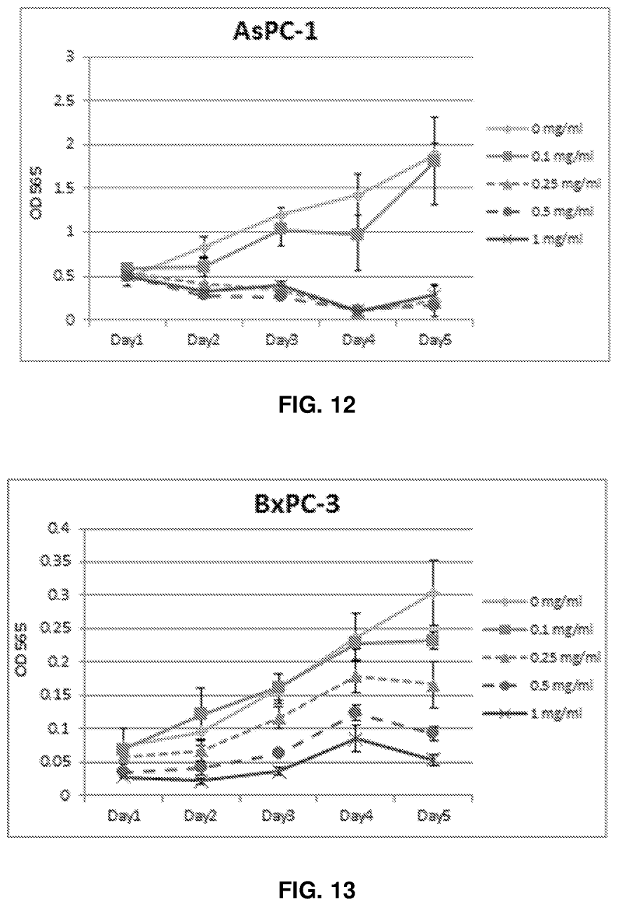

FIG. 12 is a graph showing the growth rate of AsPC-1 cells cultured in the presence of a composition of the invention at various concentrations. The AsPC-1 cell line is derived from nude mouse xenografts initiated with ascites of human with pancreatic adenocarcinoma. Mixture 1 inhibited growth of AsPC-1 cells in a concentration-dependent manner.

FIG. 13 is a graph showing the growth rate of BxPC-3 cells cultured in the presence of a composition of the invention at various concentrations. The BxPC-3 cell line is derived from human primary adenocarcinoma of the pancreas. Mixture 1 inhibited growth of BxPC-3 cells in a concentration-dependent manner.

Effects of Compositions on Molecular Weight of TAD-600.

Compositions of the invention promote formation of supramolecular polymers of TAD-600, a reduced form of glutathione. TAD-600 can exist in various polymeric forms.

FIG. 14 is graph showing results from electrospray mass spectrometric (ES-MS) analysis of a monomeric form of TAD-600.

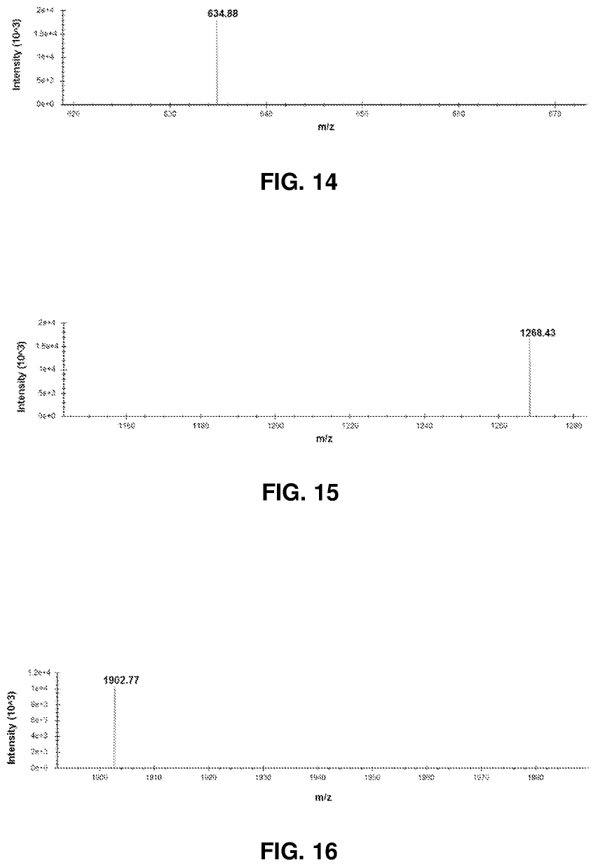

FIG. 15 is graph showing results from ES-MS analysis of a dimeric form of TAD-600.

FIG. 16 is graph showing results from ES-MS analysis of a trimeric form of TAD-600.

The average molecular weight of TAD-600 was analyzed after incubation in the presence of the monomer tetra aqua diglycine ferrous hexa aqua ferrous bis sulfate. Table 1 shows the average molecular weight and average degree n of three samples of TAD-600. Samples 1 and 2 are control samples, and sample 3 was manufactured to continuously grow its molecular weight overtime when dissolved in water. Sample 3 was analyzed after incubation for 20 minutes or three days.

TABLE 1

| | | | Sample 3, | Sample 3, |
|---|---|---|---|---|
| | Sample 1 | Sample 2 | 20 minutes | 3 days |
| Ave. MW (Da) | 10110 | 5609 | 1585 | 6372 |
| Ave. Degree n | 15.9 | 8.8 | 2.5 | 10.1 |

Effects of Compositions on Potency of TAD-600.

The TAD-600 samples described above in relation to Table 1 were analyzed for their ability to inhibit growth of cancer cell lines.

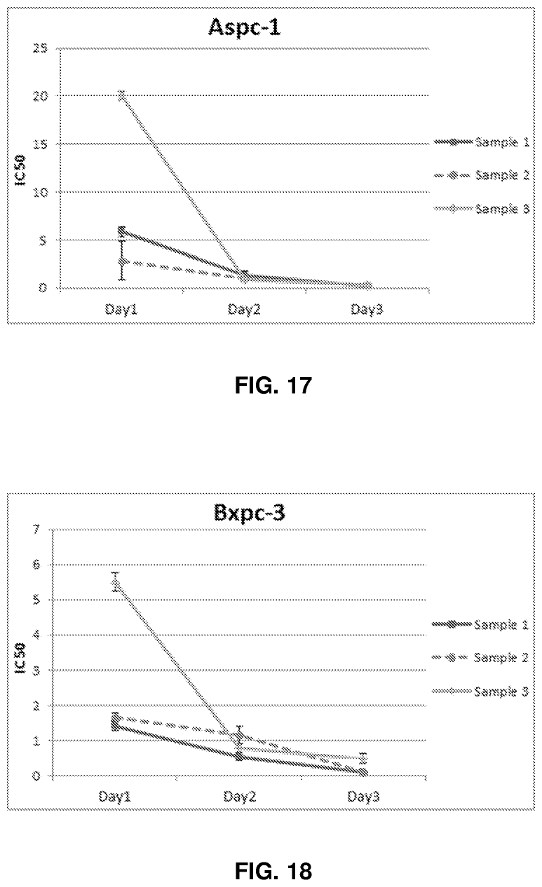

FIG. 17 shows the effect of different samples of TAD-600 on inhibition of growth of AsPC-1 cells. TAD-600 samples were tested after allowing particles to grow for 1 day, 2 days, and 3 days. Sample three showed an increase in efficacy, i.e., a decrease in IC₅₀, over time, which correlated with an increase in particle size.

FIG. 18 shows the effect of different samples of TAD-600 on inhibition of growth of BxPC-3 cells. TAD-600 samples were tested after allowing particles to grow for 1 day, 2 days, and 3 days. Sample three showed an increase in efficacy, i.e., a decrease in IC₅₀, over time, which correlated with an increase in particle size.

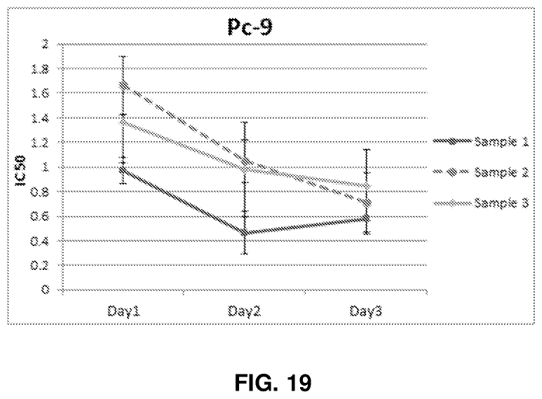

FIG. 19 shows the effect of different samples of TAD-600 on inhibition of growth of PC-9 cells. TAD-600 samples were tested after allowing particles to grow for 1 day, 2 days, and 3 days. Sample three showed an increase in efficacy, i.e., a decrease in IC₅₀, over time, which correlated with an increase in particle size.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A composition of formula (I):
[M(C₂H₅NO₂)₂(H₂O)₄][M(H₂O)₆](SO₄)₂, (I)
wherein:
M is a metal; and
the [M(C₂H₅NO₂)₂(H₂O)₄] group and the [M(H₂O)₆] group are linked to each other via a SO₄ that forms a bond with an H₂O molecule from the [M(C₂H₅NO₂)₂(H₂O)₄] group and a bond with an H₂O molecule from the [M(H₂O)₆] group.

2. The composition of claim 1, wherein M is not chelated by (C₂H₅NO₂) or SO₄.

3. The composition of claim 1, wherein M is a metal other than Co, Mg, Mn, Ni, and Zn.

4. The composition of claim 1, wherein M has an oxidation state of +2.

5. The composition of claim 4, wherein M is Fe.

6. A composition comprising:
a compound of formula (II):
X.MSO₄.5H₂O (II)
wherein:
X is selected from the group consisting of a first amino acid, a peptide, and a protein; and
M is a metal that is not chelated by X or SO₄; and
a second compound selected from the group consisting of Y, MSO₄.4H₂O, and MSO₄.H₂O, wherein Y is a second amino acid.

7. The composition of claim 6, wherein M has an oxidation state of +2.

8. The composition of claim 7, wherein M is selected from the group consisting of Ca, Co, Cr, Cu, Fe, Mg, Mn, Ni, Se, and Zn.

9. The composition of claim 8, wherein M is Fe.

10. The composition of claim 6, wherein X is glycine.

11. The composition of claim 10, wherein;
the second compound is Y;
the second amino acid is glycine; and
Y is alpha-form glycine or gamma-form glycine.

12. The composition of claim 9, wherein:
X and the second amino acid are glycine;
the second compound is ferrous sulfate tetrahydrate; and
the composition further comprises:
ferrous sulfate monohydrate;
alpha-form glycine; and
gamma-form glycine.

13. The composition of claim 6, further comprising arginine or citrulline.

14. A composition comprising:
a compound comprising a metal, sulfate, and an amino acid-containing component selected from the group consisting of an amino acid, a peptide, and a protein; and
arginine or citrulline.

15. The composition of claim 14, wherein the metal is not chelated by the amino acid-containing component or the sulfate.

16. The composition of claim 15, wherein the metal has an oxidation state of +2.

17. The composition of claim 16, wherein the metal is selected from the group consisting of Ca, Co, Cr, Cu, Fe, Mg, Mn, Ni, Se, and Zn.

18. The composition of claim 17, wherein the metal is Fe.

19. The composition of claim 14, wherein the amino acid-containing component is glycine.

20. The composition of claim 18, wherein:
the amino acid-containing component is glycine; and
the composition comprises:
ferrous sulfate tetrahydrate;
ferrous sulfate monohydrate;
alpha-form glycine; and
gamma-form glycine.

21. A method of treating a condition in a subject, the method comprising providing a composition comprising a compound comprising a metal, a sulfate, and an amino acid-containing component selected from the group consisting of a first amino acid, a peptide, and a protein, wherein the metal is not chelated by the amino acid-containing component or the sulfate.

22. The method of claim 21, wherein the compound has a formula (II):
X.MSO₄.5H₂O (II)
wherein:
X is the amino acid-containing component; and
M is the metal that is not chelated by the amino acid-containing component or the sulfate.

23. The method of claim 21, wherein the composition further comprises a second compound selected from the group consisting of MSO₄.4H₂O, MSO₄.H₂O, a second amino acid, arginine, and citrulline, wherein M is the metal.

24. The method of claim 21, wherein the metal has an oxidation state of +2.

25. The method of claim 24, wherein the metal is selected from the group consisting of Ca, Co, Cr, Cu, Fe, Mg, Mn, Ni, Se, and Zn.

26. The method of claim 25, wherein the metal is Fe.

27. The method of claim 21, wherein the amino acid-containing component is glycine.

28. The composition of claim 23, wherein:
the second amino acid is glycine; and
the second compound is alpha-form glycine or gamma-form glycine.

29. The method of claim 26, wherein
the amino acid-containing component is glycine; and
the composition further comprises:
ferrous sulfate tetrahydrate;
ferrous sulfate monohydrate;
alpha-form glycine; and
gamma-form glycine.

30. The method of claim 29, wherein the composition further comprises arginine or citrulline.
